(19) United States
(12) Patent Application Publication
(10) Pub. No.: US 2023/0226281 A1
(43) Pub. Date: Jul. 20, 2023

(54) Drug Delivery Device Recognition

(71) Applicant: Sanofi, Paris (FR)

(72) Inventor: Michael Helmer, Frankfurt am Main (DE)

(21) Appl. No.: 18/011,362

(22) PCT Filed: Jun. 23, 2021
(86) PCT No.: PCT/EP2021/067150

§ 371 Date: Dec. 19, 2022

(30) Foreign Application Priority Data
Jun. 25, 2020 (EP) 20315321.8

(51) Int. Cl. A61M 5/315; A61M 5/00

(52) U.S. Cl. CPC A61M 5/31568; A61M 5/002; A61M 2205/3306; A61M 2205/587

(57) ABSTRACT

A system includes a drug delivery device, a user device including a camera, and a case for the user device. The case includes a slot adapted to receive the drug delivery device, as well as a first wall, a second wall, and two side walls defining a space therebetween that is configured to receive the user device. When the user device and the drug delivery device are inserted into the case, the case is configured to align the drug delivery device and the camera of the user device such that the camera can capture images of at least one feature of the drug delivery device from one or both of a predefined distance and a predefined orientation.

### CROSS REFERENCE TO RELATED APPLICATIONS

The present application is the national stage entry of International Patent Application No. PCT/EP2021/067150, filed on Jun. 23, 2021, and claims priority to Application No. EP 20315321.8, filed on Jun. 25, 2020, the disclosures of which are incorporated herein by reference.

### TECHNICAL FIELD

The present disclosure relates to a drug delivery device recognition and devices and systems used in drug delivery device recognition.

### BACKGROUND

Pen-type drug delivery devices have application where regular injection by users (patients) themselves occurs. This is increasingly common among patients having diabetes where self-treatment enables such patients to conduct effective management of their diabetes.

A drug delivery device may be for example a pre-filled disposable insulin pen. Alternatively, a re-usable pen may be used. A re-usable pen allows replacement of an empty drug cartridge by a new one. Either pen may come with a set of needles that are replaced before each use. The insulin dose to be injected can then for instance be manually selected at the insulin pen by turning a dosage dial and observing the actual dose from a dosage window of the insulin pen. The dose is then injected by inserting the needle into a suitable skin portion and pressing an injection button of the insulin pen.

It may be desirable to provide a user with additional or continued assistance in use of the drug delivery device. For example, the user may need to use more than one drug delivery device, and may struggle to recognize or remember when to use which device.

It may also be advantageous to monitor the status of the device and/or the correct use of the device by the user, either permanently or temporarily (e.g. when the user is still not familiar with the device).

In certain situations, it may be advantageous to verify the device and confirm that the device is authentic (i.e. not e.g. a counterfeiting device).

### SUMMARY

According to a first aspect, a system consisting of a drug delivery device, a user device and a case for the user device is provided, the case comprising a first wall, a second wall and at least two side walls, the walls defining a space therebetween, the space being configured to receive a user device, the case further comprising a slot adapted to receive a drug delivery device; the user device comprising at least one camera; wherein, when the user device and the drug delivery device are inserted into the case, the case is configured to align the camera of the user device and the drug delivery device such that the camera is able to capture images of at least one feature of the drug delivery device from a predefined distance and/or orientation.

In a second aspect, a case for a user device is provided, the case comprising a first wall, a second wall and at least two side walls, the walls defining a space therebetween, the space being configured to receive a user device; and a slot adapted to receive a drug delivery device; wherein, when the user device and the drug delivery device are inserted into the case, the case is adapted to align a camera of the user device and the drug delivery device such that the camera is able to capture images of at least one feature of the drug delivery device from a predefined distance and/or orientation.

In a third aspect, a method of recognizing a drug delivery device is provided, the drug delivery device comprising at least one distinctive feature, the method comprising: inserting the drug delivery device into a case; inserting a user device into the case; wherein the case configured to align a camera of the user device and the drug delivery device such that the camera is able to capture images the drug delivery device from a predefined distance and/or orientation; the method further comprising: capturing, using a camera of a user device, an image of the drug delivery device; identifying, based on the captured image, using a processor of the user device, one or more distinctive features of the drug delivery device; comparing, using the processor of the user device, the identified distinctive features to predefined distinctive features stored in a memory of the user device; identifying, based on the comparison, the drug delivery device; outputting, using a screen of the user device, the result of the identification.

In a fourth aspect, a method of calculating a dose of a medicament administered by a drug delivery device is provided, the drug delivery device comprising a container for a medicament, the container comprising a bung for displacing the medicament, the method comprising: inserting the drug delivery device into a case; inserting a user device into the case; wherein the case configured to align a camera of the user device and the drug delivery device such that the camera is able to capture images the drug delivery device from a predefined distance and/or orientation; the method further comprising: capturing, using the camera of the user device, a first image of the container; determining, from the first image, using a processor of the user device, a first position of the bung, the first position corresponding to a position of the bung before a dose of a medicament is administered; capturing, using the camera of the user device, a second image of the container; determining, from the second image, using a processor of the user device, a second position of the bung, the second position corresponding to a position of the bung after the dose of the medicament is administered; determining, based on the first and second positions, the dose administered.

Embodiments of the first, second, third and fourth aspects are provided in the dependent claims and in the detailed description.

### BRIEF DESCRIPTION OF THE FIGURES

Specific embodiments will now be described, by way of example only, with reference to the accompanying drawings in which:

FIG. 1 is a schematic view of the system of a drug delivery device, a user device and a case for the user device.

FIG. 2 shows a perspective view of the drug delivery device.

FIGS. 3-4 show the drug delivery device with example distinctive features.

FIGS. 5 and 6 show an example of a drug container with a bung.

FIG. 7 shows an example of a container with a bung as seen through a drug window of the drug delivery device.

FIG. 8 is a schematic view of a user device.

FIG. 9 is a flow chart showing a method of recognizing a device. FIG. 10 is a flow chart showing a method of recognizing administered dose.

FIG. 11 is a perspective view of a user device case.

FIGS. 12 and 13 are front views of a user device case.

### DETAILED DESCRIPTION

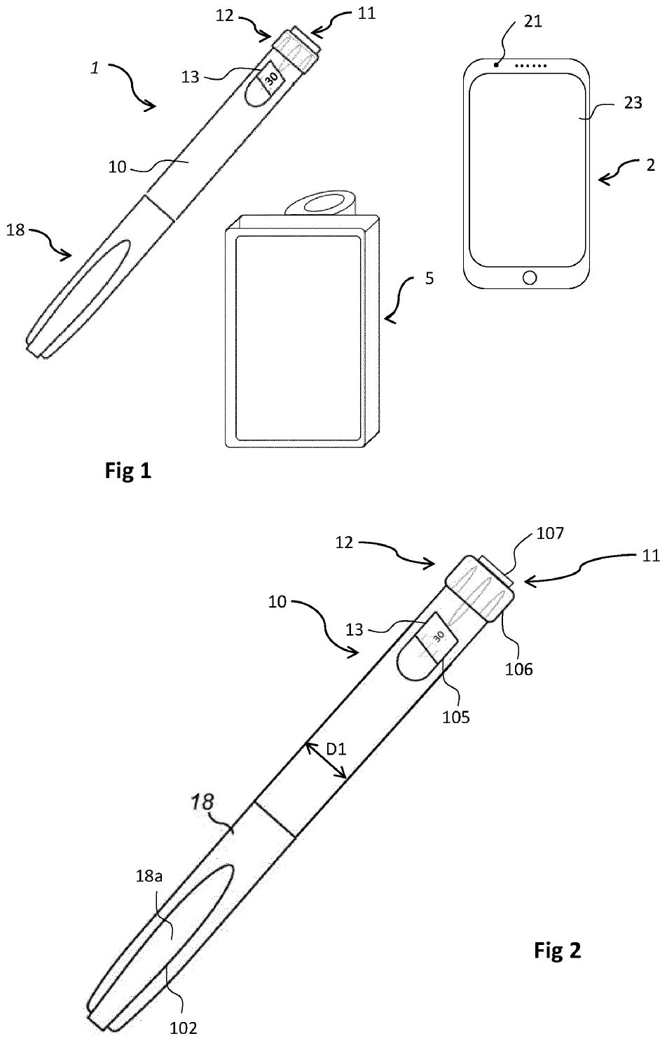

An exemplary system according to the present disclosure, as seen in FIG. 1, comprises a drug delivery device 1, a user device 2 and a case 5 for accommodating the user device 2 and the drug delivery device 1.

The drug delivery device 1 may be e.g. an insulin pen. The drug delivery device may be any other pen-type injection device. The user device 2 may be e.g. a mobile phone 2 or a tablet. The user device 2 has a camera 21 and a display 23 associated with the user device 2. The user device 2 also has at least one rear camera (not shown). The user device 2 may further comprise a flash light (not shown) positioned close to (e.g. next to) the at least one rear camera.

FIG. 2 is a view of an example drug delivery device 1. An example of such device is Sanofi's Solostar™ insulin injection pen. The drug delivery device 1 may be any suitable pen-type or auto-injector-type drug delivery device.

The drug delivery device 1 comprises a housing 10. In general, the housing 10 is provided with an injection mechanism, e.g. an injection button 11, a dosage dial 12, a dosage window 13, and a container region 14. A needle (not shown) can be affixed to the container region 14. The housing is further provided with a cap 18, which covers the container region 14 when the device 1 is not in use (and the needle is not attached). Before the needle is affixed to the housing 1, the needle is protected by an inner needle cap (not shown) and an outer needle cap (not shown).

The housing 10 is provided with a container region 14 to which the needle can be affixed. The container region 14 contains a container. The container contains the drug to be injected. The drug may be e.g. insulin. The container may be e.g. an insulin container.

The container region 14 may comprise a drug window 14a. The drug window 14a is for example an opening covered in glass, clear plastic or other transparent material through which the user can see the container and/or the contents of the container).

An appropriate dose of a drug can be selected by turning the dosage dial 12. Turning the dosage dial 12 may cause the dosage dial 12 to extend helically away from the body 10 of the drug delivery device 1 along the longitudinal axis of the body 10. The extension may correspond to the dose dialled (described below).

Turning the dosage dial 12 may cause a mechanical clicker to provide haptic and acoustic feedback to a user. The numbers displayed in dosage window 13 are present on a sleeve by printing and the sleeve is contained in housing 10 and mechanically interacts with a piston in the insulin container, which is positioned in the container region 14. When needle is stuck into a skin portion of a patient and then injection button 11 is pushed, the insulin dose displayed in dosage window 13 will be ejected from the injection device 1. When the needle of the injection device 1 remains for a certain time in the skin portion after the injection button 11 is pushed, a high percentage of the dose is injected into the patient's body. Ejection of the insulin dose also causes a mechanical click sound, which is different from the sounds produced when using dosage dial 12.

The selected dose is displayed via dosage window 13, for instance an insulin dose in multiples of so-called International Units (IU), wherein one IU is the biological equivalent of about 45.5 micrograms of pure crystalline insulin (1/22 mg). An example of a selected dose displayed in dosage window 13 may, for instance, be 30 IU, as shown in FIGS. 1-4.

The user device 2 may contain suitable software (e.g. a suitable application) which assists the user in handling the drug delivery device 1. For example, the user device 2 may contain suitable software which is able to recognize a particular drug delivery device 1 and then, based on the recognition of the drug delivery device 1, provide the user with assistance. Recognition of the drug delivery device 1 is described below. Alternatively or in addition, the software may be able to recognize features related to the administered dose (described below), and assist user in providing feedback on dosing of the medicament, logging the administered dose, and the like.

For example, the user device 2 may be able to provide or enable one or more of the following functions: indicate to the user whether the user has selected a correct drug delivery device 1; advise the user on the status of the drug delivery device 1 (e.g. advise the user in case a component of the drug delivery device 1 is not functional, or not fully functional); advise the user on the status of the drug loaded in the drug delivery device 1 (e.g. in case the drug is or appears to be past its expiration date, the drug is cloudy when it is supposed to be clear, etc.); indicate whether the drug delivery device 1 is authentic (i.e. not a counterfeiting device); verify the dose administered by the drug delivery device 1; etc.

The drug delivery device 1 has one or more distinctive features. The user device 2 stores suitable software enabling the user device 2 to identify the distinctive features and compare them to a pre-determined set of distinctive features associated with a particular device. The user device 2 therefore enables recognition of the drug delivery device 1. The distinctive features may enable the user device 2 to identify the status of the drug delivery device 1.

The user device 2 may store more than one distinctive feature of a given drug delivery device 1. If the user device 2 stores more than one distinctive feature of a given drug delivery device 1, the drug delivery device 1 may be identified by the user device 2 with more accuracy. If the user device 2 stores more than one distinctive feature of a given drug delivery device 1, it may be possible to recognize one or more of the following: position of the drug delivery device 1, orientation of the drug delivery device 1 with respect to the user device 2, distance of the drug delivery device 1 from the user device 2, etc. Alternatively or in addition, if the user uses the user device 2 for capturing a video rather than a static picture, it may be possible to identify the movements of the drug delivery device 1, changes in its position, orientation, distance from the user device 2 etc.

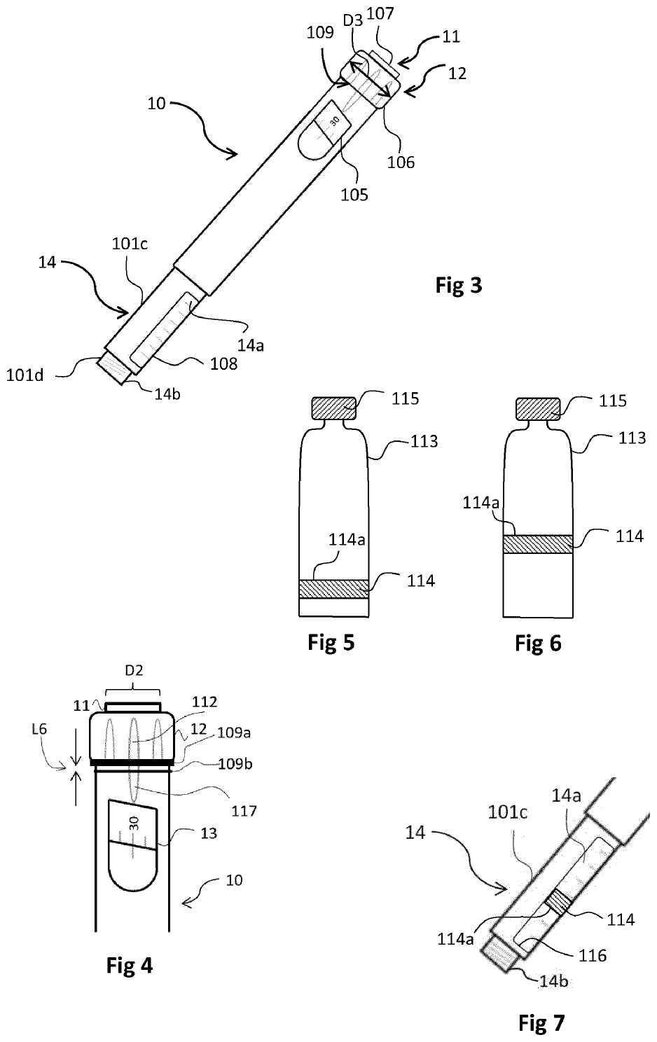

Example features which may serve as distinctive features are shown in FIG. 3. The distinctive features may be for example:

outer shape 101c, 101d of the body 10 of the container region 14 (i.e. with the cap 18 removed);

color of the of the body 10 of the drug delivery device 1;

the shape and/or color 105 of the dosage window 13;

the shape and/or color 106 of the dosage dial 12;

the shape and/or color 108 of the drug window 14a;

the distance L6 between the dosage dial 12 and the body 10 of the drug delivery device 1;

number and/or position of notches 112 on the dosage dial 12;

the diameter D1 of the body 10 of the drug delivery device 1 (see FIG. 3);

the diameter D2 of the injection button 11;

the diameter D3 of the dosage dial 12 (see FIG. 4);

the ratio of any combination of the length L6 and diameters D1-D3 (in general, any of the ratios Dx/Dy, or L6/Dy, where x, y stand for the number of the respective length or diameter);

distances (not shown) between any two of the distinctive features listed above and/or ratios of any two of these distances.

The distinctive features may be used alone or in combination. The user device 2 may recognize one or more of the features in a given drug delivery device 1. Any one of the features listed above can be used for drug delivery device recognition in combination with any other of the features. The user device 2 may store any subset of the above listed distinctive features, including all the distinctive features. Recognition by the user device 2 of any of the distinctive features listed above can be combined with recognition by the user device 2 of any other of the distinctive features. The drug delivery device 1 may be provided with any subset of the above listed distinctive features, including all the distinctive features. A drug delivery device 1 may be provided with any one of the features listed above in combination with any other of the features.

The user device 2 stores predefined distinctive features of a given drug delivery device 1 (such as any one of the distinctive features, a subset of the distinctive features listed above, or all of the distinctive features listed above). The user device 2 is capable of comparing the distinctive features measured from the image or video captured by the camera 21 to the predefined distinctive features stored in the memory 25 of the user device 2 to identify which drug delivery device 1 is presented to the camera 21.

For example, the user may own two different drug delivery devices. Each of these drug delivery devices has a set of distinctive features. The first set of distinctive features of the first drug delivery device differs in at least one of the distinctive features, and preferably in more than one distinctive feature, from the second set of distinctive features of the second drug delivery device. The user device 2 stores the first set and the second set of distinctive features of the first and second drug delivery device, respectively. When the user presents one of their drug delivery devices to a camera (the front camera 21 or the rear camera) of the user device 2, the user device 2 captures an image of the drug delivery device presented to the camera. Based on the distinctive features, the user device 2 uses the captured image to derive the distinctive features of the drug delivery device 1 shown to the camera. The user delivery device 1 compares the distinctive features derived from the image with the stored first and second sets of distinctive features. The user device 2 then identifies which one of the first and the second set matches the distinctive features derived from the image, and determines which one of the user-owned drug delivery devices has been presented to the camera.

The diameter D1 of the body 10 of the drug delivery device 1 may be measured in the widest point of the drug delivery device 1. Alternatively, the diameter D1 may be measured in a pre-defined location, for example along the line of contact between the body 10 and the cap 18, along the line where the dosage dial 12 is positioned, or in any other suitable location.

The diameter D2 of the injection button 11 may be measured at the outer-most region of the injection button 11. Alternatively, the diameter D2 may be measured in the widest point of the injection button 11.

Diameter D3 of the dosage dial 12 may be measured at one of the ends (outer-most regions) of the dosage dial 12. The diameter D3 may be measured along the collar 109a. Alternatively, the diameter D2 may be measured in the widest point of the dosage dial 12.

The recognition of the drug delivery device 1 based on the recognition of the shapes of various other features (e.g. the dosage window 13, the dosage dial 12, the injection button 11 or the drug window 14a) may depend on the distance of the drug delivery device 1 from a camera of the user device 2.

The recognition of the drug delivery device 1 based on the recognition of the dimensions of parts of the drug delivery device 1 (such as the length L6 and the diameters D1 to D3) may depend on the distance of the drug delivery device 1 from the user device 2.

FIGS. 5 and 6 show an example of a container 113 that may be used with the drug delivery device 1. It will be appreciated that the container 113 may be replaced by any suitable alternative. For example, a single-use pen-type drug delivery device may use an integrated container rather than a container shown in FIGS. 5 and 6. Container 113 may be replaceable in the drug delivery device 1.

Regardless of the type of container used, the container 113 may comprise a bung 114 and a closure 115. The closure engages the needle via a needle interface (not shown). The bung 114 closes a first or distal end of the container 113. The closure 115 closes a second or proximal end of the container 113.

The bung 114 is movable within the container 113. Movement of the bung 114 pushes the medicament contained in the container 113 out into the needle and out of the drug delivery device 1. FIG. 5 shows an example configuration of the container 113 before a dose is administered. FIG. 6 shows an example configuration of the container 113 after a dose is administered. FIGS. 5 and 6 show that after the dose is administered, the bung 114 is moved from a first position closer to the distal end of the container 113 into a second position further from the distal end and closer to the proximal end of the container 113.

During use of the drug delivery device 1, the container is contained in the container region 14. The container 113 and the bung 114 may be observed through a drug window 14a. The position of the bung 114 and/or the change of position of the bung 114 may be observed through the drug window 14a.

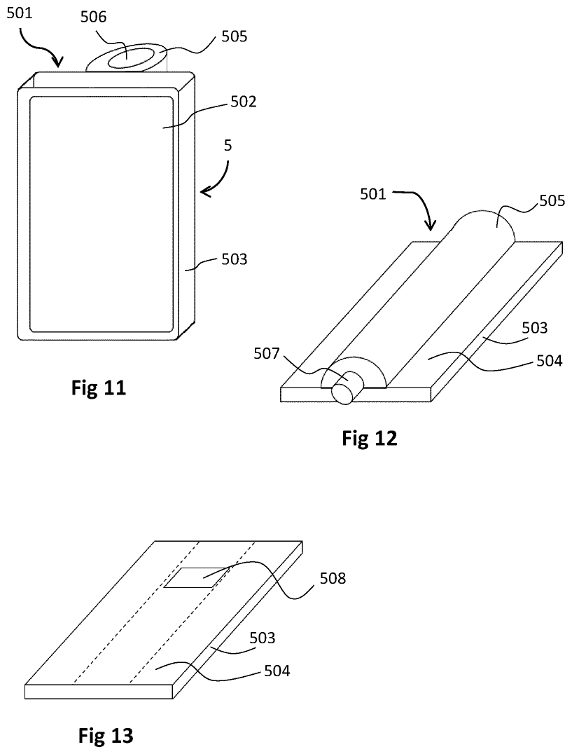

One way of establishing a known distance between the user device 2 and the drug delivery device 1 and a known orientation of the drug delivery device 1 with respect to the user device 2 is using a specially designed case 5 (shown in detail FIGS. 11-13). The case 5 has a first wall 502, a second wall 504 and at least two sidewalls 503. The first wall 502, the second wall 504 and the sidewalls 503 define a space 501 for accommodating the user device 2. The second wall 504 of the case 5 comprises a slot 505. The slot 505 may be for example a tube, as shown in FIGS. 11 and 12, but other shapes are possible. The slot 505 has an inside space 506 adapted to accommodate the drug delivery device 1. The slot 505 may further comprise an extension 507. The extension 507 may be adapted to accommodate the needle mount 14b. The extension 507 may be adapted to accommodate the dosage dial 12 and the button 11.

The second wall 504 of the case 5 comprises a lens system 508. The lens system 508 is positioned to be aligned with the camera of the user device 2. The lens system 508 may enhance the measurement of the features of the drug delivery device 1. Preferably, the lens system compensates for the short distance between the camera and the drug delivery device 2 when the drug delivery device 2 is positioned in the slot 505 of the case 5.

In an embodiment, the lens system 508 also comprises a light guide (not shown). Preferably, the light guide is configured to lead light from the flash light to the drug delivery device 1, thus improving the resolution of the captured images and/or recognition of the distinctive features. In an embodiment, the light guide may help recognition of the bung 114 and/or its position. Thus, the light guide may improve precision of recognition of the administered dose.

In an embodiment, the case 5 may be configured such that the user device 2 may be positioned in the case 5 facing the first wall 502. The front wall 502 may be adapted to allow the user to see and/or operate the screen 23 (e.g. a touch screen) of the user device 2. In this case, the lens system 508 is positioned to be aligned with the rear camera of the user device 2.

In an embodiment, the user device 2 may be positioned in the case 5 facing the second wall 504. In this case, the lens system 508 is positioned to be aligned with the front camera 21 of the user device 2. The user device 2 may be stored such that the display 21 is not accessible to the user when the user device 2 is inserted into the case 5.

Alternatively, the case 5 may comprise a lid (not shown). The slot 505 may then be positioned on or within the lid. The lid may be adapted to be opened and closed. When the lid is open, the display screen 23 of the user device 2 is accessible to the user. When the lid is closed, the slot 505 is aligned with the front camera 21 of the user device. Once the drug delivery device 1 is stored in the slot 505, the drug delivery device 1 is also aligned with the front camera 21 of the user device 2.

The slot 505 may be positioned so as to align the lens system 508, the drug window 14a, the bung 114 and the camera of the user device 2.

When the drug delivery device 1 is inserted into the inside space 506 of the slot 505, the drug delivery device 1 is positioned in a specific position and orientation with respect to the user device 2. In particular, the slot 505 positions the drug delivery device 1 in a specific position (distance, orientation) with respect to the front camera 21 or rear camera of the user device 2. This then allows the user device 2 to capture images of the drug delivery device 1 from a known distance and angle.

Using the known distance and orientation of the drug delivery device 1 with respect to the user device 2, the user device 2 may process the captured images and measure the distinctive features described above. Alternatively or in addition, using the known distance and orientation of the drug delivery device 1 with respect to the user device 2, the user device 2 may process the captured images and measure the position of the bung 114 before and after the dose is administered, respectively, and derive the administered dose from the measurement.

Using the poach 5, the measurement of the distinctive features and/or the administered dose may be more precise due to the known position (distance, orientation) of the drug delivery device 1 from the camera of the user device 2.

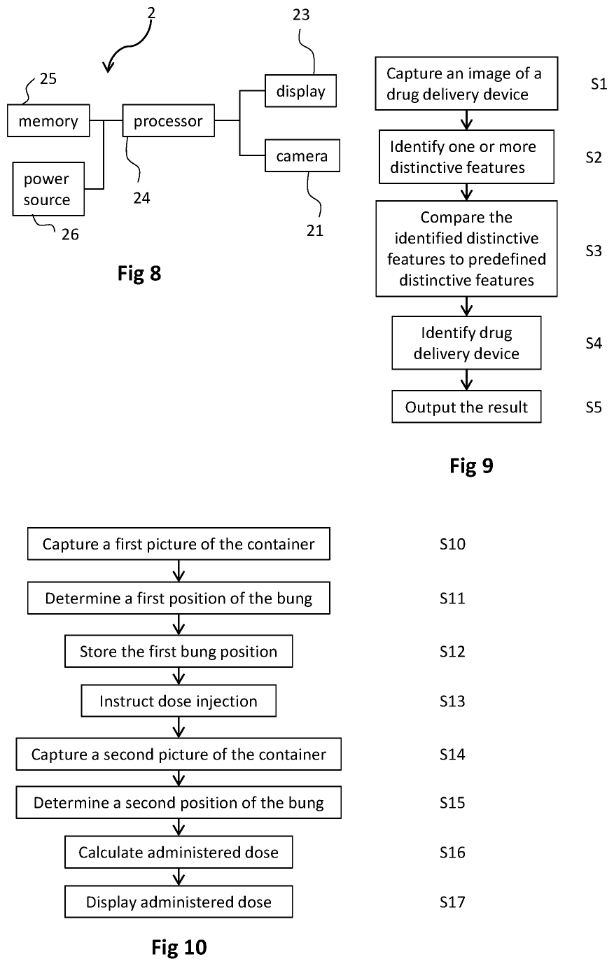

The user device 2 (shown schematically in FIG. 8) comprises a processor 24, a memory 25, at least one camera 21, a display 23, and a power source 26. The memory 25 stores suitable software (e.g. a software application) for the drug delivery device 1 recognition, as described above. The processor 24 executes the software in accordance with what has been described above.

In use, the user captures an image of a drug delivery device 1, using the camera 21 of the user device 2 (FIG. 9, step S1). The captured image is then processed by the user device 2, and one or more distinctive features are identified in the captured image (step S2). The identified distinctive features are compared with distinctive features stored in the user device 2 (step S3). The drug delivery device is then identified (step S4), and the result is output (step S5).

The above-described devices and methods may be employed for example in a method illustrated in FIG. 10 (described below).

The drug delivery device 1 is first positioned in the slot 505 of the case 5. The user device 2 is also positioned in the case 5, in the space 501. Preferably, the user device 2 stores a set of parameters of the drug delivery device 2 in its memory.

After the user device 2 and the drug delivery device 1 are positioned in the case 5, the user device 2 may capture a first picture of the container 113 (step S10) of the drug delivery device 1.

From the first picture of the drug delivery device 1 and/or from the first picture of the container 113, a first position of the bung 114 may be determined (step S11). The first position of the bung 114 typically corresponds to a position of the bung 114 before a dose is administered.

The user device 2 stores the first bung position (step S12).

The user device 2 may instruct the user to inject the dose (step S13). The user device 2 may optionally require a confirmation from the user that the dose has been injected.

The user device 2 may capture a second picture of the container 113 (step S14).

From the second picture of the drug delivery device 1 and/or from the second picture of the container 113, a second position of the bung 114 may be determined (step S15).

The second position of the bung 114 typically corresponds to a position of the bung 114 after a dose is administered. From the known (pre-stored) parameters of the cartridge 113 and the known first and second positions of the bung 114, the administered dose may be calculated (step S16).

Optionally, the administered dose may be displayed on the display 23 of the user device 2 (step S17). Alternatively or in addition, information on whether a correct dose has been administered may be displayed. For example, if it is determined that the dose corresponds to the (pre-stored or pre-selected) user's recommended dose, a confirmation that a correct dose has been administered may be displayed.

To aid the bung position recognition, the container 113 and/or the bung 114 and/or the drug window 14a may comprise at least one contrast feature. For example, the inner face of the bung 114a may be provided in a contrast color and/or with a contrast lining. Similarly, the drug window 14a may have at least one of its boundaries 116 (shown in FIG. 7) provided in a contrast color.

It will be clear to the skilled person that a similar contrast feature may be provided on the container (e.g. in a position close to the proximal end, positioned such that after insertion of the container into the container region, this contrast feature is visible through the drug window 14a). In this context, contrast color is a color which is visible towards the other features and/or the drug contained in the container. For example, the container region 14 may be provided in a dark color (such as black, grey, dark green or brown), the bung 114 may also be provided in a dark color (same or different from the container region 14 color), while the contrast features may be provided in a transparent color (yellow, orange) or in a light color (such as white, beige, yellow or red).

With the case 5, as described above, the images captured by the user device 2 during the course of the method of FIG. 10 are associated with a known position of the drug delivery device 1 with respect to user device 2. Therefore, the identification of the drug delivery device 1 and/or the measurement of the administered dose do not require any additional steps of positioning the drug delivery device 1 with respect to the user device, deriving the precise position (distance and orientation) of the drug delivery device 1 with respect to the user device. Thus the respective methods are simplified. In addition, the identification of the drug delivery device 1 and/or the measurement of the administered dose may be more precise than without the case 5.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs.

For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body.

For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codeable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N-(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrome.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab')2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present disclosure include, for example, Fab fragments, F(ab')2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present disclosure, which encompass such modifications and any and all equivalents thereof.

An example drug delivery device may involve a needle-based injection system as described in Table 1 of section 5.2 of ISO 11608-1:2014(E). As described in ISO 11608-1:2014(E), needle-based injection systems may be broadly distinguished into multi-dose container systems and single-dose (with partial or full evacuation) container systems. The container may be a replaceable container or an integrated non-replaceable container.

As further described in ISO 11608-1:2014(E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014(E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014(E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

1-14. (canceled)

15. A system comprising:
a drug delivery device;
a user device comprising a camera; and
a case for the user device, the case comprising:
a slot adapted to receive the drug delivery device, and
a first wall, a second wall, and two side walls defining a space therebetween that is configured to receive the user device,
wherein, when the user device and the drug delivery device are inserted into the case, the case is configured to align the drug delivery device and the camera of the user device such that the camera can capture images of at least one feature of the drug delivery device from one or both of a predefined distance and a predefined orientation.

16. The system of claim 15, wherein the case further comprises a lens system that is positioned between the camera and the drug delivery device when the user device and the drug delivery device are inserted into the case.

17. The system of claim 15, wherein the user device further comprises a flash light and the case further comprises a light guide, the light guide being configured to guide a light from the flash light towards the drug delivery device.

18. The system of claim 15, wherein the drug delivery device comprises a container for containing a medicament, and wherein the container comprises a bung for displacing the medicament.

19. The system of claim 18, wherein the case is configured to align the camera and the container such that the camera is positioned to capture an image of the bung.

20. The system of claim 19, wherein the user device is configured to:
capture a first image of the container using the camera; and
determine a first position of the bung from the first image using a processor, the first position corresponding to a reference position of the bung before a dose of the medicament has been administered.

21. The system of claim 20, wherein the user device is further configured to:
capture a second image of the container using the camera; and
determine a second position of the bung from the second image using the processor, the second position corresponding to an adjusted position of the bung after the dose of the medicament has been administered.

22. The system of claim 21, wherein the user device is further configured to determine a size of the dose based on the first and second positions.

23. The system of claim 15, wherein the camera comprises a back camera.

24. The system of claim 23, wherein the case is configured to align the back camera and the drug delivery device such that the back camera can capture the images of the at least one feature of the drug delivery device from one or both of the predefined distance and the predefined orientation.

25. The system of claim 15, wherein the camera comprises a front camera.

26. The system of claim 25, wherein the case is configured to align the front camera and the drug delivery device such that the front camera can capture the images of the at least one feature of the drug delivery device from one or both of the predefined distance and the predefined orientation.

27. The system of claim 15, wherein the case comprises a lid, and wherein the slot is provided on the lid.

28. A case for a user device, the case comprising:
a first wall, a second wall, and two side walls defining a space therebetween that is configured to receive a user device; and
a slot adapted to receive a drug delivery device,
wherein the case is adapted to align the drug delivery device and a camera of the user device when the user device and the drug delivery device are inserted into the case such that the camera can capture images of at least one feature of the drug delivery device from one or both of a predefined distance and a predefined orientation.

29. The case of claim 28, wherein the case further comprises a lens system that is positioned between the camera and the drug delivery device when the user device and the drug delivery device are inserted into the case.

30. The case of claim 28, wherein the case further comprises a light guide that is configured to guide a light from a flash light of the user device towards the drug delivery device when the user device and the drug delivery device are inserted into the case.

31. The case of claim 28, wherein the case is configured to align the camera and a container for containing a medicament such that the camera is positioned to capture an image of a bung configured to displace the medicament.

32. A method of recognizing a drug delivery device that comprises a distinctive feature, the method comprising:
inserting the drug delivery device into a case;
inserting a user device into the case, wherein the case is configured to align a camera of the user device and the drug delivery device such that the camera can capture images of the drug delivery device from one or both of a predefined distance and a predefined orientation;
capturing an image of the drug delivery device using the camera of the user device;
identifying the distinctive feature of the drug delivery device based on the image using a processor of the user device;
comparing the distinctive feature to a set of predefined distinctive features stored in a memory of the user device to generate a comparison;
identifying the drug delivery device based on the comparison; and
outputting a result comprising an identity of the drug delivery device using a screen of the user device.

33. The method of claim 32, wherein the distinctive feature comprises an outer shape, a color, a diameter, a distance between two components, a number of components, a position of components, or a ratio of components.

34. The case of claim 32, wherein the case comprises a lens system that is positioned between the camera and the drug delivery device when the user device and the drug delivery device are inserted into the case.
